(19) United States
(12) Patent Application Publication
(10) Pub. No.: US 2020/0397683 A1
(43) Pub. Date: Dec. 24, 2020

(54) HAIR DYE KIT

(71) Applicant: Henkel AG & Co. KGaA, Duesseldorf (DE)

(72) Inventors: TORSTEN LECHNER, Langenfeld (DE); JUERGEN SCHOEPGENS, Schwalmtal (DE); MARC NOWOTTNY, Moenchengladbach (DE)

(73) Assignee: Henkel AG & Co. KGaA, Duesseldorf (DE)

(21) Appl. No.: 16/977,083

(22) PCT Filed: Feb. 7, 2019
(86) PCT No.: PCT/EP2019/052974

§ 371 Date: Aug. 31, 2020

(30) Foreign Application Priority Data
Mar. 14, 2018 (DE) 10 2018 203 837.6

(51) Int. Cl. A61K 8/58; A61K 8/34; A61K 8/46; A61K 8/36; A61K 8/362

(52) U.S. Cl. CPC A61K 8/585; A61K 8/342; A61K 8/463; A61K 2800/882; A61K 8/362; A61K 2800/432; A61K 2800/43; A61K 8/36

(57) ABSTRACT

The object of the present disclosure is a multi-component packaging unit (kit-of-parts) for dyeing keratinous fibers, in particular human hair, which contains separately from one another
a first container containing a first cosmetic preparation (A) and
a second container containing a second cosmetic preparation (B),
   where
the first cosmetic preparation (A) contains at least one specific organic silicon compound corresponding to formula (I)

(R₁O)ₐ(R₂)_{b}Si-(A)_{c}-[NR₃-(A′)]_{d}-[O-(A″)]ₑ-[NR₄-(A′″)]_{f}-Si(R₂′)_{b′}(OR₁′)_{a′} (I), and
the second cosmetic preparation (B) contains water.

A second subject-matter of the present disclosure is a process for dyeing hair using the preparations (A) and (B) described above.

### CROSS-REFERENCE TO RELATED APPLICATION

This application is a U.S. National-Stage entry under 35 U.S.C. § 371 based on International Application No. PCT/EP2019/052974, filed Feb. 7, 2019, which was published under PCT Article 21(2) and which claims priority to German Application No. 10 2018 203 837.6, filed Mar. 14, 2018, which are all hereby incorporated in their entirety by reference.

### TECHNICAL FIELD

The subject-matter of the present application is preparations for changing the color of keratinous fibers, human hair, comprising at least two separately made-up preparations (A) and (B). The first preparation (A) contains at least one special organic silicon compound and is preferably made up essentially water-free. The second preparation (B) contains water.

Another subject of this application is a process for coloring human hair using two preparations (A) and (B).

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dying with good fastness properties and good Grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the dye into the hair fiber. Compared to oxidative hair dyeing, the dying obtained with direct dyes have a shorter shelf life and quicker wash ability. Dyeing with direct dyes usually remain on the hair for a period of between about 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. They can therefore be removed again without residue by a few washes with detergents containing surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

### BRIEF SUMMARY

Multi-component packaging units for dyeing keratinous fibers and method for dyeing human hair are provided herein. In an embodiment, a multi-component packaging unit for dyeing keratinous fibers includes, separately from one another, a first container comprising a first cosmetic preparation (A) and a second container comprising a second cosmetic preparation (B). The first cosmetic preparation (A) comprises at least one organic silicon compound corresponding to formula (I)

(R₁O)ₐ(R₂)_{b}Si-(A)_{c}-[NR₃-(A′)]_{d}-[O-(A″)]ₑ-[NR₄-(A′″)]_{f}-Si(R₂′)_{b′}(OR₁′)_{a′} (I),
where
R1, R1′, R1″, R2, R2′ and R2″ independently of one another represent a C₁-C₆ alkyl group,
A, A′, A″, and A′″ independently of one another represent a linear or branched divalent C₁-C₂₀ alkylene group,
R₃ and R₄ independently represent a hydrogen atom, a C₁-C₆ alkyl group, a hydroxy C₁-C₆ alkyl group, a C₂-C₆ alkenyl group, an amino C₁-C₆ alkyl group or a group of formula (II)

(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″,
c is 0 or 1,
d is 0 or 1,
e stands for 0 or 1,
f stands for 0 or 1,
A″″ represents a linear or branched divalent C₁-C₂₀ alkylene group,
provided that at least one of c, d, e, and f is different from 0.
   The second cosmetic preparation (B) comprises water.

In another embodiment, a method for dyeing human hair includes the following steps in the order indicated: (1) providing a cosmetic preparation (A), (2) providing a cosmetic preparation (B), (3) mixing cosmetic preparations (A) and (B), (4) applying the application mixture prepared in step (3) to the hair, (5) allowing the application mixture act on the hair and (6) rinsing off the application mixture.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The purpose of the present disclosure was to provide a dyeing system with fastness properties comparable to those of oxidative dyeing. Wash fastness properties should be outstanding, but the use of oxidation dye precursors normally used for this purpose should be avoided. A technology was sought that would allow the direct dyes and pigments known from state-of-the-art technology to be fixed to the hair in an extremely permanent manner.

Surprisingly, it has now turned out that the above-mentioned task can be excellently solved if a multi-component packaging unit is used for dyeing the keratin fibers, which provides the user with two separately packaged preparations (A) and (B). The cosmetic composition (A) contains at least one reactive organic silicon compound of formula (I). To prevent premature abreaction, this preparation (A) is preferably made up without water. The second preparation (B) is a component containing water. Immediately before use, the user mixes preparations (A) and (B). When the organic silicon compound comes into contact with water, a polymerization or oligomerization reaction is set in motion. In this way, the coloring compound contained in preparation (A) and/or (B), which may for example be a direct dye or a pigment, can be embedded in a polymeric environment and deposited in the form of a film on the keratin fiber. The film produced “in situ” in this way, in which the coloring compound is embedded, is exemplified by outstanding wash fastness.

A first object of the present disclosure is a multi-component packaging unit (kit-of-parts) for dyeing keratin fibers, human hair, which contains separately from one another
a first container containing a first cosmetic preparation (A) and
a second container containing a second cosmetic preparation (B),
   where
the first cosmetic preparation (A) contains at least one organic silicon compound corresponding to formula (I)

(R₁O)ₐ(R₂)_{b}Si-(A)_{c}-[NR₃-(A′)]_{d}-[O-(A″)]ₑ-[NR₄-(A′″)]_{f}-Si(R′₂)_{b′}(OR₁′)_{a′} (1),

where
R1, R1′, R1″, R2, R2′ and R2″ independently of one another represent a C₁-C₆ alkyl group,
A, A′, A″, A′″ and A″″ independently of one another represent a linear or branched divalent C₁-C₂₀ alkylene group
R₃ and R₄ independently represent a hydrogen atom, a C₁-C₆ alkyl group, a hydroxy C₁-C₆ alkyl group, a C₂-C₆ alkenyl group, an amino C₁-C₆ alkyl group or a group of formula (II)

(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″,
c is 0 or 1,
d is 0 or 1,
e stands for 0 or 1,
f stands for 0 or 1,
provided that at least one of c, d, e, and f is different from 0, and
the second cosmetic preparation (B) contains water.

### Agent for Dyeing Keratinous Fibers

Keratinic fibers, keratin containing fibers or keratin fibers are to be understood as furs, wool, feathers, and human hair. Although the agents as contemplated herein are primarily suitable for dyeing keratin fibers, there is nothing in principle to prevent their use in other areas.

The term “coloring agent” is used in the context of the present disclosure for a coloring of the keratin fibers, in particular the hair, caused by the use of direct dyes and/or color pigments, the aforementioned coloring compounds being deposited in a film on the surface of the keratin material. The film is formed in situ by oligomerization or polymerization of the organic silica compound or compounds of formula (I) initiated by their contact with water.

Due to the high resistance of the formed film, the produced dyeing's have an extremely good water resistance and wash fastness.

### Multi-Component Packaging Unit

The multi-component packaging unit (kit-of-parts) as contemplated herein comprises at least two containers, the first container containing a first cosmetic preparation (A) and the second container containing the second cosmetic preparation (B). These two products are different cosmetic products, each containing all essential ingredients in a cosmetic carrier.

The preparation (A) is preferably formulated with a low water content or preferably anhydrous and can, for example, be in powder form or as a paste. Also, the packaging based on an oily or fatty carrier is as contemplated herein.

The preparation (B), on the other hand, is watery or contains water. The preparation (B) can be formulated based on an aqueous or aqueous-alcoholic carrier, for example. Suitable forms of packaging are for example a cream, an emulsion, a gel, or a surfactant-containing foaming solution such as a shampoo or foam aerosol.

Shortly before use, the user mixes preparations (A) and (B) to produce the ready-to-use dye which is applied to the keratinous fibers or hair.

In addition to the first and second container, the kit-of-parts as contemplated herein may also include other containers with additional components. For example, a third, separately packaged container can optionally be included, which contains a pre-treatment agent, a conditioner, or a shampoo, for example.

Within the scope of a further version, a multi-component packaging unit (kit-of-parts) for dyeing keratinic fibers, human hair, is thus preferred, which contains separately from one another
a first container containing a first cosmetic preparation (A) and
a second container containing a second cosmetic preparation (B) and
a third container containing a third cosmetic preparation (C), wherein
   the first cosmetic preparation (A) contains at least one organic silicon compound corresponding to formula (I)

(R₁O)ₐ(R₂)_{b}Si-(A)_{c}-[NR₃-(A′)]_{d}-[O-(A″)]ₑ-[NR₄-(A′″)]_{f}-Si(R₂′)_{b′}(OR₁′)_{a′} (I),

where
R₁, R₁, R₁″, R₂, R₂′ and R₂″ independently represent a C₁-C₆ alkyl group,
A, A′, A″, A′″ and A″″ independently of one another represent a linear or branched divalent C₁-C₂₀ alkylene group
R₃ and R₄ independently represent a hydrogen atom, a C₁-C₆ alkyl group, a hydroxy C₁-C₆ alkyl group, a C₂-C₆ alkenyl group, an amino C₁-C₆ alkyl group or a group of formula (II)

(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″,
c is 0 or 1,
d is 0 or 1,
e stands for 0 or 1,
f stands for 0 or 1,
provided that at least one of c, d, e, and f is different from 0, and
the second cosmetic preparation (B) contains water, and
the third cosmetic preparation (C) is an agent for cleaning and/or care of the keratinous fibers.

### Preparation (A) in the First Container

The preparation (A) in the first container is exemplified by its content of at least one organic silicon compound corresponding to formula (I)

(R₁O)ₐ(R₂)_{b}Si-(A)_{c}-[NR₃-(A′)]_{d}-[O-(A″)]ₑ-[NR₄-(A′″)]_{f}-Si(R₂′)_{b′}(OR₁′)_{a′} (I),

where
R1, R1′, R1″, R2, R2′ and R2″ independently of one another represent a C₁-C₆ alkyl group,
A, A′, A″, A′″ and A″″ independently of one another represent a linear or branched divalent C₁-C₂₀ alkylene group
R₃ and R₄ independently represent a hydrogen atom, a C₁-C₆ alkyl group, a hydroxy C₁-C₆ alkyl group, a C₂-C₆ alkenyl group, an amino C₁-C₆ alkyl group or a group of formula (II)

(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″,
c is 0 or 1,
d is 0 or 1,
e stands for 0 or 1,
f stands for 0 or 1,
provided that at least one of c, d, e, and f is different from 0.

Organic silicon compounds, alternatively called organosilicon compounds, are compounds which either have a direct silicon-carbon bond (Si—C) or in which the carbon is bonded to the silicon atom via an oxygen, nitrogen, or sulfur atom.

The organosilicon compounds of the formula (I) as contemplated herein carry the silicon-containing groups (R₁O)ₐ(R₂)_{b}Si— and —Si(R′₂)_{b′}(OR₁′)_{a′} at each of their two ends.

The central part of the molecule contains the groupings -(A)_{c}-und —[NR₃-(A′)]_{d}- und —[O-(A″)]ₑ- and —[NR₄-(A′″)]_{f}- Here, each of c, d, e and f may independently represent the number 0 or 1, with the proviso that at least one of c, d, e and f is different from 0. In other words, an organic silicon compound of formula (I) as contemplated herein contains at least one grouping from the group of -(A)- and —[NR₃-(A′)]- and —[O-(A″)]- and —[NR₄-(A′″)]-.

The substituents R₁, R₁′, R₁″, R₂, R₂′ and R₂″, A, A′, A″, A′″ and A″″, R₃ and R₄ and R4 of the compounds of formula (I) are exemplified below: Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆-alkenyl group are vinyl, allyl, but-2-enyl, but-3-enyl and isobutenyl, preferred C₂-C₆-alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl, a 5-hydroxypentyl and a 6-hydroxyhexyl group; a 2-hydroxyethyl group is particularly preferred. Examples of an amino-C₁-C₆-alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂) and the butylene group (—CH₂—CH₂—CH₂—CH₂—). The propylene group (—CH₂—CH₂—CH₂) is particularly preferred. From a chain length of 3 C atoms, the divalent alkylene groups can also be branched. Examples of branched divalent C₃-C₂₀ alkylene groups are (—CH₂—CH(CH₃)—) and (—CH₂—CH(CH₃)—CH₂—

In the two terminal structural units (R₁O)ₐ(R₂)_{b}Si— and —Si(R₂′)b′(O_{R}1′)a′ the radicals R1, R2, R1′ and R2′ independently of one another represent a C₁-C₆ alkyl group. R1, R2, R1′ and R2′ independently of one another represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Analogously, a′ stands for an integer from 1 to 3, and b′ stands for the integer 3-a′. If a′ stands for the number 3, then b′ is equal to 0. If a′ stands for the number 2, then b is equal to 1. If a′ stands for the number 1, then b′ is equal to 2.

Dyeing's with the best wash fastness values could be obtained if the residues a and a′ both stand for the number 3. In this case b and b′ both stand for the number 0.

A multi-component packaging unit is particularly preferred within the scope of a further version, which is exemplified in that the cosmetic preparation (A) contains at least one organic silicon compound of the formula (I), where
R1 and R1′ independently represent a methyl group or an ethyl group,
a and a′ both stand for the number 3 and
b and b′ both stand for the number 0.

If a and a′ both stand for the number 3 and b and b′ both stand for the number 0, the organic silicon compound of the present disclosure corresponds to formula (Ia)

(R₁O)₃Si-(A)_{c}-[NR₃-(A′)]_{d}-[O-(A″)]ₑ-[NR₄-(A′″)]_{f}-Si(OR₁′)₃ (Ia).

The radicals c, d, e, and f can independently of one another stand for the number 0 or 1, whereby at least one of c, d, e, and f is different from zero.

The radicals or abbreviations c, d, e and f thus define which of the groups -(A)- and —[NR₃-(A′)]_{d}- and —[O-(A″)]ₑ- and —[NR₄-(A′″)]_{f}- is located in the middle part of the organic silicon compound of the formula (I) (or of the formula (Ia)).

In this context, the presence of certain groupings has proved to be particularly beneficial in terms of increasing washability. Particularly good results were obtained when at least two of the residues c, d, e, and f stand for the number 1. C and d both stand for the number 1. Furthermore, e and f both stand for the number 0.

When c and d both stand for the number 1 and e and f both stand for the number 0, the organic silicon compound of the present disclosure corresponds to formula (Ib)

(R₁O)ₐ(R₂)_{b}Si-(A)-[NR₃-(A′)]-Si(R₂′)_{b′}(OR₁′)_{a′} (Ib).

A multi-component packaging unit (kit-of-parts) for dyeing keratin fibers, human hair, is also very particularly preferred, which contains separately from one another
a first container containing a first cosmetic preparation (A) and
a second container containing a second cosmetic preparation (B), wherein
the first cosmetic preparation (A) contains at least one organic silicon compound corresponding to formula (Ib)

(R₁O)ₐ(R₂)_{b}Si-(A)-[NR₃-(A′)]-Si(R₂′)_{b′}(OR₁′)_{a′} (Ib).

where
R₁, R₁′, R₁″, R₂, R₂′ and R₂″ independently represent a C₁-C₆ alkyl group,
A, A′ and A″″ independently of one another represent a linear or branched divalent C₁-C₂₀ alkylene group,
R₃ represents a hydrogen atom, a C₁-C₆ alkyl group, a hydroxy-C₁-C₆ alkyl group, a C₂-C₆ alkenyl group, an amino-C₁-C₆ alkyl group or a group of formula (II)

(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″, and
the second cosmetic preparation (B) contains water.

The radicals A, A′, A″, A′″ and A″″ independently of one another represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently of one another represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂) or a butylene group (—CH₂—CH₂—CH₂—CH₂-). In particular, the radicals A, A′, A″, A′″ and A″″ stand for a propylene group (—CH₂—CH₂—CH₂—).

If the radical d represents the number 1, then the organic silicon compound of formula (I) as contemplated herein contains a structural grouping [NR₃-(A′)].

If the radical f represents the number 1, then the organic silicon compound of formula (I) as contemplated herein contains a structural grouping —[NR₄-(A′″)].

Here, the radicals R₃ and R₄ independently of one another represent a hydrogen atom, a C₁-C₆-alkyl group, a hydroxy-C₁-C₆-alkyl group, a C₂-C₆-alkenyl group, an amino-C₁-C₆-alkyl group or a grouping of the formula (II)

(A″″)-Si(R₂″)_{b}″(OR₁″)ₐ″ (II),

Very preferably the radicals R₃ and R₄ independently of one another represent a hydrogen atom, a methyl group, a 2-hydroxyethyl group, a 2-alkenyl group, a 2-aminoethyl group or a grouping of the formula (II).

If the radical d represents the number 1 and the radical f represents the number 0, the organic silicon compound as contemplated herein contains the grouping [NR₃-(A′)] but not the grouping —[NR₄-(A′″)]. If the radical R₃ now stands for a grouping of the formula (II), the cosmetic preparation (A) contains an organic silicon compound with 3 reactive silane groups.

A multi-component packaging unit which is particularly preferred within the scope of a further version is exemplified in that the cosmetic preparation (A) contains at least one organic silicon compound of formula (I)

where
c and d both stand for the number 1,
e and f both stand for the number 0,
A and A′ independently of one another represent a linear C₁-C₆ alkylene group and
   R₃ represents a hydrogen atom, a methyl group, a 2-hydroxyethyl group, a 2-alkenyl group, a 2-aminoethyl group or a group of formula (II).

A multi-component packaging unit which is particularly preferred within the scope of a further version is exemplified in that the cosmetic preparation (A) contains at least one organic silicon compound of formula (I)

where
c and d both stand for the number 1,
e and f both stand for the number 0,
A and A′ independently of one another represent a methylene group (—CH₂—), an ethylene group (—CH₂—CH₂—) or a propylene group (—CH₂—CH₂—CH₂), and
R3 represents a hydrogen atom, a methyl group, a 2-hydroxyethyl group, a 2-alkenyl group, a 2-aminoethyl group or a group of formula (II).

A multi-component packaging unit which is particularly preferred in the context of a further version is exemplified in that the cosmetic preparation (A) contains at least one organic silicon compound of formula (I)

where
c and d both stand for the number 1,
e and f both stand for the number 0,
A and A′ independently of one another represent a methylene group (—CH₂—), an ethylene group (—CH₂—CH₂—) or a propylene group (—CH₂—CH₂—CH₂), and
R3 represents a methyl group, a 2-hydroxyethyl group, a 2-alkenyl group, a 2-aminoalkyl group or a group of formula (II).

In a further preferred design, a multi-component packaging unit as contemplated herein is exemplified in that the first cosmetic preparation (A) contains at least one organic silicon compound of formula (I) which is selected from

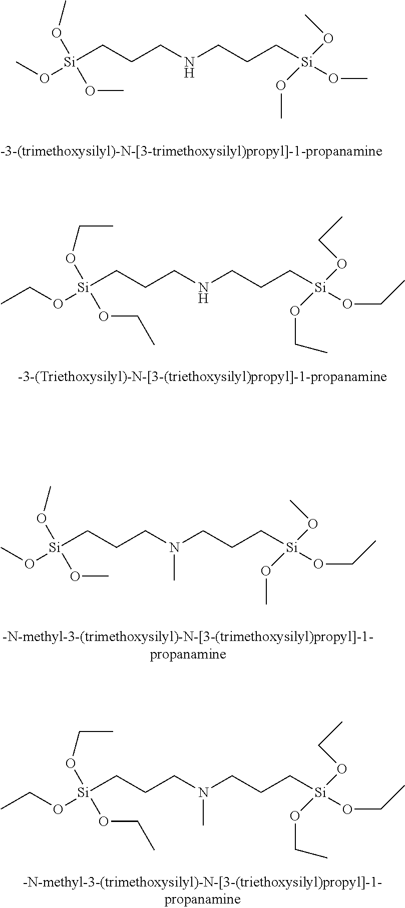

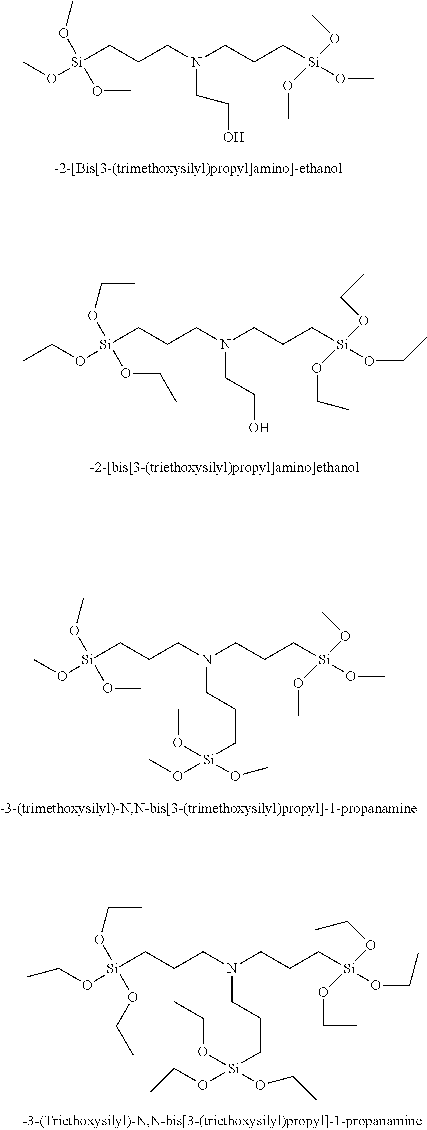

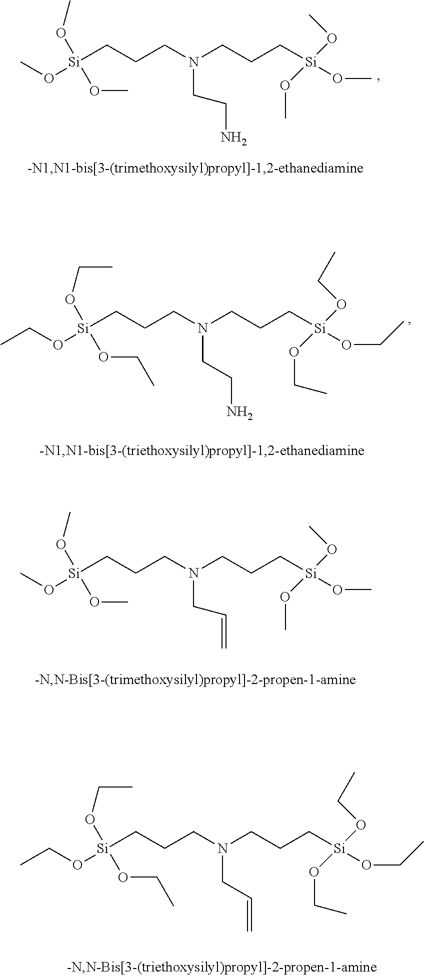

The organic silicon compounds are commercially available.

For example, Bis(trimethoxysilylpropyl)amines with the CAS number 82985-35-1 can be purchased from Sigma-Aldrich.
Bis[3-(triethoxysilyl)propyl]amine with the CAS number 13497-18-2 can be purchased from Sigma-Aldrich, for example.
N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.
3-(triethoxysilyl)-N,N-bis[3-(triethoxysilyl)propyl]-1-propanamine with the CAS number 18784-74-2 can be purchased for example from Fluorochem or Sigma-Aldrich.

In the context of a further design explicitly particularly preferred is a multi-component packaging unit which is exemplified in that the cosmetic preparation (A) contains at least one organic silicon compound of the formula (I) which is selected from the group of
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

The organic silicon compounds of the formula (I) are reactive compounds which are made up separately in the preparation (A) and are only mixed with a water-containing preparation (B) shortly before use.

In this context, it has proved to be particularly preferable to choose a relatively high concentration of the organic silicon compound(s) of formula (I) in the preparation (A). In this way, undesired side reactions or unwanted reactions with other ingredients of the carrier formulation can be suppressed. Preferably the cosmetic preparation (A) contains—based on the total weight of the preparation (A)—one or more organic silicon compounds corresponding to formula (I) in a total quantity of from about 20 to about 100% by weight, preferably of from about 35 to about 100% by weight, more preferably of from about 45 to about 100% by weight, still more preferably of from about 55 to about 100% by weight and very particularly preferably of from about 65 to about 100% by weight.

A multi-component packaging unit is particularly preferred in a further version, exemplified in that the cosmetic preparation (A) contains—based on the total weight of the preparation (A)—one or more organic silicon compounds of the formula (I) in a total amount of from about 20 to about 100% by weight, preferably from about 35 to about 100% by weight, more preferably from about 45 to about 100% by weight, still more preferably from about 55 to about 100% by weight and very particularly preferably from about 65 to about 100% by weight.

The organic silicon compounds of formula (I) react on contact with water under hydrolysis and oligomerization or polymerization. This reaction ensures the formation of a very stable and durable film on the keratinous fibers, which encloses the coloring substances contained in the product and thus enables the creation of extremely washable colors.

It has proven to be explicitly preferred if this oligomerization or polymerization takes place as a controlled reaction at a defined point in time shortly before the application of the ready-to-use agent. For this purpose, preparation (A) is particularly preferred to be low in water, or very preferably water-free.

Within the framework of a further version, a multi-component packaging unit is particularly preferred, which is exemplified in that the first cosmetic preparation (A) is essentially anhydrous and preferably—based on the total weight of the preparation (A)—contains less than about 5.0% by weight, more preferably less than about 2.5% by weight, still more preferably less than about 1.0% by weight and very preferably less than about 0.1% by weight of water.

The preparation (A) may be in the form of a powder, for example. It is also possible to make the preparation (A) available as a paste or oil. In this case, the organic silicon compounds of formula (I) may be incorporated into an inert cosmetic carrier. Fatty components have proven to be suitable as inert cosmetic carriers.

As contemplated herein, fat constituents are organic compounds with a solubility in water at room temperature (22° C.) and atmospheric pressure (760 mmHg) of less than about 1% by weight, preferably less than about 0.1% by weight. The definition of fat constituents explicitly covers only uncharged (i.e. non-ionic) compounds. Fatty components have at least one saturated or unsaturated alkyl group with at least 8 C atoms. The molecular weight of the fat constituents is a maximum of about 5000 g/mol, preferably a maximum of about 2500 g/mol and particularly preferably a maximum of about 1000 g/mol. The fat components are neither polyoxyalkylated nor polyglycerylated compounds.

In this context, preferred fat constituents are defined as the constituents from the group of C₈-C₃₀ fatty alcohols, C₈-C₃₀ fatty acid triglycerides, C₈-C₃₀ fatty acid monoglycerides, C₈-C₃₀ fatty acid diglycerides and/or hydrocarbons. For the purposes of the present disclosure, only non-ionic substances are explicitly regarded as fat components. Charged compounds such as fatty acids and their salts are not considered to be fat components.

C₈-C₃₀ fatty alcohols can be saturated, mono- or polyunsaturated, linear, or branched fatty alcohols with from bout 8 to about 30 C atoms.

Examples of preferred C₈-C₃₀ linear saturated fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol).

Preferred linear unsaturated fatty alcohols are (9Z)-octadec-9-en-1-ol(oleyl alcohol), (9E)-octadec-9-en-1-ol(elaidyl alcohol) (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-Eicos-9-en-1-ol), arachidone alcohol ((5Z,8Z,11Z,14Z)-Eicosa-5,8,11,14-tetraen-1-ol), erucyl alcohol ((13Z)-Docos-13-en-1-ol) and/orbrassidyl alcohol ((13E)-Docosen-1-ol).

The preferred representatives for branched fatty alcohols are 2-octyl-dodecanol, 2-hexyl-dodecanol and/or 2-butyl-dodecanol.

For the purposes of this present disclosure, a C₈-C₃₀ fatty acid triglyceride is understood to be the triesters of trivalent alcohol glycerol with three equivalents of fatty acid. Both structurally identical and different fatty acids within a triglyceride molecule can be involved in the formation of esters.

As contemplated herein, fatty acids are saturated or unsaturated, unbranched, or branched, unsubstituted, or substituted C₈-C₃₀ carboxylic acids. Unsaturated fatty acids can be mono- or polyunsaturated. For an unsaturated fatty acid, its C—C double bond(s) may have the Cis or Trans configuration.

Fatty acid triglycerides are exemplified by their particular suitability, in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], Palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] and/or nervonic acid [(15Z)-tetracos-15-enoic acid].

The fatty acid triglycerides can also be of natural origin. The fatty acid triglycerides or mixtures thereof occurring in soybean oil, peanut oil, olive oil, sunflower oil, macadamia nut oil, moringa oil, apricot kernel oil, marula oil and/or optionally hardened castor oil are particularly suitable for use in the kit-of-parts as contemplated herein.

A C₈-C₃₀ fatty acid monoglyceride is the monoester of the trivalent alcohol glycerol with one equivalent of fatty acid. Either the middle hydroxy group of glycerol or the terminal hydroxy group of glycerol may be esterified with the fatty acid.

The C₈-C₃₀ fatty acid monoglyceride is exemplified by its special suitability, in which a hydroxy group of the glycerol is esterified with a fatty acid, whereby the fatty acids are selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], Palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] or nervonic acid [(15Z)-tetracos-15-enoic acid].

A C₈-C₃₀ fatty acid diglyceride is the diester of the trivalent alcohol glycerol with two equivalents of fatty acid. Either the middle and one terminal hydroxy group of glycerol may be esterified with two equivalents of fatty acid, or both terminal hydroxy groups of glycerol are esterified with one fatty acid each. The glycerol can be esterified with two structurally identical fatty acids or with two different fatty acids.

Fatty acid diglycerides are exemplified by their special suitability, in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], Palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, elaeostearic acid [(9Z,11E,13E)-octadeca-9,11,3-trienoic acid], arachidonic acid [(5Z,8Z,11Z,14Z)-cosa-5,8,11,14-tetraenoic acid] and/or nervonic acid [(15Z)-tetracos-15-enoic acid].

As contemplated herein, the term C₈-C₃₀ fatty acid esters refers to esters of C₈-C₃₀ fatty acids with aliphatic C₁-C₁₀ alcohols. The C₁-C₁₀ alcohols can be linear. From a chain length of 3 C atoms, the alcohols can also be branched. Particularly suitable C₈-C₃₀ fatty acid esters can be selected from the group of isopropyl myristate (myristic acid isopropyl ester) and isopropyl stearate (stearic acid isopropyl ester).

Hydrocarbons are compounds consisting exclusively of the atoms carbon and hydrogen with from about 8 to about 80 C atoms. In this context, aliphatic hydrocarbons such as mineral oils, liquid paraffin oils (e.g. Paraffinum Liquidum or Paraffinum Perliquidum), isoparaffin oils, semi-solid paraffin oils, paraffin waxes, hard paraffin (Paraffinum Solidum), Vaseline and polydecenes are particularly preferred.

Liquid paraffin oils (Paraffinum Liquidum and Paraffinum Perliquidum) have proven to be particularly suitable in this context. Paraffinum Liquidum, also known as white oil, is the preferred hydrocarbon. Paraffinum Liquidum is a mixture of purified, saturated, aliphatic hydrocarbons, mainly including hydrocarbon chains with a C-chain distribution of from about 25 to about 35 C-atoms.

A multi-component packaging unit is particularly preferred in the context of another version, exemplified in that the cosmetic preparation (A) contains one or more fat constituents from the group of C₈-C₃₀ fatty alcohols, C₈-C₃₀ fatty acid triglycerides, C₈-C₃₀ fatty acid monoglycerides, C₈-C₃₀ fatty acid diglycerides, C₈-C₃₀ fatty acid esters and/or hydrocarbons.

It has been found that C₈-C₃₀ fatty alcohols and hydrocarbons are particularly suitable as inert cosmetic carriers.

A multi-component packaging unit is particularly preferred within the framework of a further version, which is exemplified in that the cosmetic preparation (A) contains one or more fat constituents from the group of C₈-C₃₀ fatty alcohols and/or hydrocarbons.

The multi-component packaging unit as contemplated herein is used for the dyeing of keratinous fibers, therefore the ready-to-use mixture produced from the preparations (A) and (B) contains at least one coloring compound, which is particularly preferably selected from the group of direct dyes and/or pigments.

The provision of the colored connections can be done in different ways. Within the scope of a design form, the coloring compounds can be packaged together with the organic silicon compound(s) in preparation (A).

A joint packaging of organic silicon compound and coloring compound is particularly suitable if the coloring compound is poorly soluble in water but readily soluble in the fatty components that can be used as an inert carrier material.

In a further embodiment, a multi-component packaging unit is particularly preferred, which is exemplified in that the first cosmetic preparation (A) contains at least one coloring compound from the group of direct dyes and/or pigments.

A multi-component packaging unit is particularly preferred in the context of a further version, which is exemplified in that the first cosmetic preparation (A) contains at least one colorant compound from the group of pigments.

Direct dyes are dyes that are applied directly to the hair and do not require an oxidative process to develop the color. Direct dyes are usually nitrophenylenediamines, nitroaminophenols, azo dyes, anthraquinones, triarylmethane dyes or indophenols.

Direct dyes can be divided into anionic, cationic, and non-ionic direct dyes, which are selected and used by the professional according to the requirements of the carrier base.

Preferred anionic direct dyes are the compounds known under the international names or trade names Bromphenol Blue, Tetrabromophenol Blue, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52.

Preferred cationic direct dyes are Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Yellow 87, Basic Orange 31 and Basic Red 51.

Non-ionic direct dyes are particularly suitable as non-ionic direct dyes, such as nitro and quinone dyes and neutral azo dyes. The preferred non-ionic direct dyes are those under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

The direct dyes may be used in an amount of from about 0.001 to about 20% by weight, of from about 0.05 to about 5% by weight, each based on the total weight of the preparation (A). The total amount of direct dyes in the preparation (A) is preferably not more than about 5% by weight.

Pigments within the meaning of the present disclosure are coloring compounds which have a solubility in water at 20° C. of less than about 0.1 g/l. Water solubility can be determined, for example, by the method described below: about 0.1 g of the pigment is weighed in a beaker. A stir-fish is added. Then make up to about 1 l with distilled water (20° C.). It is stirred for an hour. If undissolved components of the pigment are still visible in the mixture after this period, the solubility of the pigment is below about 0.1 g/l.

Suitable color pigments may be of organic and/or inorganic origin. Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. In particular, preferred color pigments are black iron oxide (CI 77499), yellow iron oxide (CI 77492), red and brown iron oxide (CI 77491), manganese violet (CI 77742), ultramarine (sodium aluminum sulfo silicates, CI77007, pigment blue 29), chromium oxide hydrate (CI77289), iron blue (ferric ferrocyanides, CI77510) and/or carmine (cochineal).

As contemplated herein, colored pearlescent pigments are also particularly preferred color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

It is therefore, as contemplated herein, particularly preferred if the preparation (A) contains at least one pigment which is a colored pigment based on mica or micaceous iron oxide and which is coated with one or more metal oxides from the group of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, pigment blue 29), chromium oxide hydrate (CI 77289), chromium oxide (CI 77288) and/or iron blue (ferric ferrocyanides, CI 77510).

Examples of particularly suitable color pigments are commercially available under the trade names Rona@, Colorona®, Xirona®, Dichrona® and Timiron® from Merck, Ariabel® and Unipure® from Sensient, Prestige@ from Eckart Cosmetic Colors and Sunshine@ from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491(Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

Due to their excellent light and temperature resistance, the use of the inorganic color pigments in the agents of the present disclosure is particularly preferred. It is also preferred if the pigments used have a certain particle size. This particle size leads on the one hand to an even distribution of the pigments in the formed polymer film and on the other hand avoids a rough hair or skin feeling after application of the cosmetic product. It is therefore advantageous as contemplated herein if the at least one pigment has an average particle size D50 of from about 1.0 to about 50 μm, preferably of from about 5.0 to about 45 μm, preferably of from about 10 to about 40 μm, of from about 14 to about 30 μm. The mean particle size D50, for example, can be determined using dynamic light scattering (DLS).

The pigment or pigments may be used in an amount of from about 0.001 to about 20% by weight, from about 0.05 to about 5% by weight, each based on the total weight of the preparation (A).

### Preparation (B) in Second Container

The multi-component packaging unit as contemplated herein comprises a second container containing a second cosmetic preparation (B). The second cosmetic preparation (B) contains water.

By mixing preparations (A) and (B), the oligomerization or polymerization of the organic silicon compounds of formula (I) is initiated. The coloring compounds are incorporated into the film produced in this way on the keratinous fibers. At the same time, this cross-linking reaction also leads to an increase in viscosity in the application mixture, which ensures that the ready-to-use mixture does not drip off the fibers.

In the course of the work leading to this present disclosure, it has proved to be particularly suitable to select a relatively high amount of water in the preparation (B).

The cross-linking reaction in the application mixture can be adjusted to the optimum speed by selecting the appropriate ratio of water to organic silicon compounds. The best results were obtained when the preparation (B)—based on the total weight of the preparation (B)—contains from about 50 to about 100% by weight, preferably from about 50 to about 95% by weight, more preferably from about 60 to about 95% by weight and most preferably from about 70 to about 95% by weight of water.

A multi-component packaging unit is particularly preferred within the framework of a further version, which is exemplified in that the second cosmetic preparation (B)—based on the total weight of the preparation (B)—contains from about 50 to about 100% by weight, preferably from about 50 to about 95% by weight, more preferably from about 60 to about 95% by weight and very particularly preferably from about 70 to about 95% by weight of water.

The speed of the cross-linking reaction can also be controlled by selecting the optimum pH value. In this context, it has proved to be particularly advantageous to add at least one acid to the aqueous preparation (B).

Suitable acids can be selected from the organic and inorganic acids. Suitable organic acids are lactic acid, citric acid, tartaric acid, malic acid, 1-hydroxyethane-1,1-diphosphonic acid, 2,6-dipicolinic acid, benzoic acid, maleic acid, succinic acid, oxalic acid, ascorbic acid, phytic acid and/or gluconic acid. Suitable inorganic acids are phosphoric acid, sulfuric acid, and hydrochloric acid.

Within the framework of a further design form, a multi-component packaging unit is particularly preferred, which is exemplified in that the second cosmetic preparation (B) contains at least one acid from the group lactic acid, citric acid tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid.

Furthermore, it is particularly preferred that the second cosmetic preparation (B) has a pH value in the range from about 0.5 to about 6, preferably from about 1 to about 5, more preferably from about 1.5 to about 4 and most preferably from about 2.0 to about 3.5.

A multi-component packaging unit is particularly preferred within the framework of a further version, which is exemplified in that the second cosmetic preparation (B) has a pH value in the range from about 0.5 to about 6, preferably from about 1 to about 5, more preferably from about 1.5 to about 4 and very particularly preferably from about 2.0 to about 3.5.

The preparation (B) may also contain one or more surfactants. The use of surfactants ensures that the ready-to-use dye is completely washed out.

The term surfactants refer to surface-active substances. A distinction is made between anionic surfactants including a hydrophobic residue and a negatively charged hydrophilic head group, amphoteric surfactants, which carry both a negative and a compensating positive charge, cationic surfactants, which in addition to a hydrophobic residue have a positively charged hydrophilic group, and nonionic surfactants, which have no charges but strong dipole moments and are strongly hydrated in aqueous solution.

Zwitterionic surfactants are those surface-active compounds which carry at least one quaternary ammonium group and at least one —COO⁽⁻⁾— or —SO₃⁽⁻⁾ group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example the cocoacylaminopropyl dimethyl ammonium glycinate, and 2-alkyl-3-carboxymethyl-3-hydroxyethyl imidazolines each having 8 to 18 C atoms in the alkyl or acyl group, and the cocoacylaminoethyl hydroxyethyl carboxymethyl glycinate. A preferred zwitterionic surfactant is the fatty acid amide derivative known under the INCI designation cocamidopropyl betaine.

Ampholytic surfactants are surface-active compounds which, apart from a C₈-C₂₄ alkyl or acyl group, contain at least one free amino group and at least one —COOH— or —SO₃H group in the molecule and can form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to about 24 C atoms in the alkyl group. Typical examples of amphoteric or zwitterionic surfactants are alkylbetaines, alkylamidobetaines, amino propionates, aminoglycinate, imidazoliniumbetaines and sulfobetaines.

Particularly preferred ampholytic surfactants are the N-cocoalkylaminopropionate, the cocoacylaminoethylaminopropionate and the C₁₂-C₁₈ acylsarcosine.

The preparation (B) may additionally contain at least one non-ionic surfactant. Suitable non-ionic surfactants have been shown to be alkyl polyglycosides and alkylene oxide adducts to fatty alcohols and fatty acids with from about 2 to about 30 mol ethylene oxide per mol fatty alcohol or fatty acid. Preparations with good properties are also obtained if they contain as nonionic surfactants fatty acid esters of ethoxylated glycerol which have been reacted with at least 2 mol ethylene oxide. The nonionic surfactants are used in a total quantity of from about 0.1 to about 45% by weight, preferably from about 1 to about 30% by weight and very preferably from about 1 to about 15% by weight, based on the total weight of the preparation (B).

In addition, the preparation (B) may also contain at least one cationic surfactant. Cationic surfactants are surfactants, i.e. surface-active compounds, each with one or more positive charges. Cationic surfactants contain only positive charges. Usually these surfactants are composed of a hydrophobic part and a hydrophilic head group, the hydrophobic part usually including a hydrocarbon backbone (e.g. including one or two linear or branched alkyl chains) and the positive charge(s) being located in the hydrophilic head group. Examples of cationic surfactants are
quaternary ammonium compounds which, as hydrophobic radicals, may carry one or two alkyl chains with a chain length of from about 8 to about 28 C atoms,
quaternary phosphonium salts substituted with one or more alkyl chains with a chain length of from about 8 to about 28 C atoms or
tertiary sulfonium salts.

Furthermore, the cationic charge can also be part of a heterocyclic ring (e.g. an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of from about 0.1 to about 45% by weight, preferably from about 1 to about 30% by weight and very preferably from about 1 to about 15% by weight, based on the total weight of the preparation (B).

Furthermore, the preparation (B) may also contain at least one anionic surfactant. Anionic surfactants are surface-active agents with exclusively anionic charges (neutralized by a corresponding counter cation). Examples of anionic surfactants are fatty acids, alkyl sulfates, alkyl ether sulfates and ether carboxylic acids with from about 12 to about 20 C atoms in the alkyl group and up to 16 glycol ether groups in the molecule.

The anionic surfactants are used in a total quantity of from about 0.1 to about 45% by weight, preferably from about 1 to about 30% by weight and very preferably from about 1 to about 15% by weight, based on the total weight of the preparation (B).

As already described above, the kit-of-parts as contemplated herein is used for the dyeing of keratinic fibers, therefore the ready-to-use mixture contains at least one coloring compound, which is particularly preferably selected from the group of direct dyes and/or pigments.

In a further version, the coloring compounds can be incorporated into the water-containing preparation (B).

The incorporation of the coloring compounds into the water-containing preparation (B) is particularly suitable if the coloring compounds are readily water-soluble and dissolve better in the preparation (B) than in the preparation (A), or if the coloring compounds carry functional groups which could possibly react with the organic silicon compounds of formula (I).

A multi-component packaging unit which is exemplified in that the second cosmetic preparation (B) contains at least one colorant compound from the group of direct-acting dyes and/or pigments is particularly preferred within the framework of a further version.

A multi-component packaging unit is particularly preferred in the context of another version, exemplified in that the second cosmetic preparation (B) contains at least one colorant compound from the group of direct-acting colorants.

Suitable direct dyes and pigments have already been described—these are the same direct dyes and pigments that can be used in preparation (A).

The direct dyes and pigments may be used in an amount of from about 0.001 to about 20% by weight, of from about 0.05 to about 5% by weight, each based on the total weight of the preparation (B).

### Preparation (C) in Third Container

It is also as contemplated herein that the coloring compounds used for coloring purposes are not incorporated into either preparation (A) or preparation (B) but are made available separately in a third preparation (C).

In the context of this embodiment, a multi-component packaging unit (kit-of-parts) for dyeing keratin fibers, human hair, is particularly preferred, which contains separately from one another
a first container containing a first cosmetic preparation (A) and
a second container containing a second cosmetic preparation (B) and
a third container containing a third cosmetic preparation (C),
   where
the first cosmetic preparation (A) contains at least one organic silicon compound corresponding to formula (I),
the second cosmetic preparation (B) contains water, and
the third cosmetic preparation (C) contains at least one coloring compound from the group of direct dyes and/or pigments.

Suitable direct dyes and pigments have already been described—these are the same direct dyes and pigments that can be used in the preparation (A) or in the preparation (B).

The direct dyes and pigments may be used in an amount of from about 0.001 to about 20% by weight, of from about 0.05 to about 5% by weight, each based on the total weight of the preparation (C).

### Other Ingredients

Preparations (A) and (B)—and if necessary also preparation (C)—may also contain other active substances, auxiliaries and additives, such as fatty alcohols, non-ionic polymers such as vinylpyrrolidinone/vinyl acrylate copolymers, polyvinylpyrrolidinone, vinylpyrrolidinone/vinyl acetate copolymers, polyethylene glycols and polysiloxanes; additional silicones such as volatile or non-volatile, straight-chain, branched or cyclic, crosslinked or non-crosslinked polyalkylsiloxanes (such as dimethicones or cyclomethicones), polyarylsiloxanes and/or polyalkylarylsiloxanes, in particular polysiloxanes with organofunctional groups, such as substituted or unsubstituted amines (amodimethicones), carboxyl, alkoxy and/or hydroxyl groups (dimethicone copolymers), linear polysiloxaneA)-polyoxyalkyleneB)-block copolymers, grafted silicone polymers; cationic polymers such as quaternized cellulose ethers, polysiloxanes having quaternary groups, dimethyldiallylammoniumchloridepolymers,acrylamide-dimethyldiallylammoniumchloride copolymers, dimethylamino-ethylmethacrylate-vinylpyrrolidinone copolymers quaternized with diethyl sulfate, vinylpyrrolidinone-imidazolinium-methochloride copolymers and quaternized polyvinyl alcohol; zwitterionic and amphoteric polymers; anionic polymers such as polyacrylic acids or cross-linked polyacrylic acids; structurants such as glucose, maleic acid and lactic acid; hair conditioning compounds such as phospholipids, for example lecitin and cephalins; perfume oils, dimethylisosorbide and cyclodextrins; fiber structure-improving active substances, in particular mono-, di- and oligosaccharides such as glucose, galactose, fructose, fructose and lactose; dyes for coloring the composition; anti-dandruff active substances such as Piroctone Olamine, Zinc Omadine and Climbazol; amino acids and oligopeptides; protein hydrolysates on animal and/or vegetable basis, as well as in the form of their fatty acid condensation products or optionally anionically or cationically modified derivatives; vegetable oils; light stabilizers and UV-blockers; active ingredients such as panthenol, pantothenic acid, pantolactone, allantoin, pyrrolidinonecarboxylic acids and their salts as well as bisabolol; polyphenols, in particular hydroxycinnamic acids, 6,7-dihydroxycumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas as well as primary, secondary and tertiary phosphates; opacifiers such as latex, styrene/PVP and styrene/acrylamide copolymers; pearlescent agents such as ethylene glycol mono- and distearate and PEG-3 distearate; pigments and blowing agents such as propane-butane mixtures, N₂O, dimethyl ether, CO₂ and air.

The selection of these additional substances will be made by the specialist according to the desired properties of the agents. Regarding other optional components and the quantities of these components used, explicit reference is made to the relevant manuals known to the specialist. The additional active ingredients and auxiliary substances are preferably used in the preparations as contemplated herein in amounts of from about 0.0001 to about 25% by weight in each case, from about 0.0005 to about 15% by weight, based on the total weight of the respective preparation.

### Mixing Ratio of Preparations (A) and (B)

As described above, the ready-to-use colorant is prepared by mixing preparations (A) and (B). In principle, the preparations (A) and (B) can be mixed in different mixing ratios, such as (A)/(B) from about 1:10 to about 10:1.

To ensure convenient mixing, it may be advantageous to use preparations (A) and (B) in approximately equal quantities.

In a further preferred design, a multi-component packaging unit as contemplated herein is therefore exemplified in that the quantities of the preparation (A) in the first container and of the preparation (B) in the second container are selected in such a way that, when the application mixture is prepared—i.e. when the preparations (A) and (B) are mixed—the mixing ratio (A)/(B) is at a value of from about 1:10 to about 10:1, preferably from about 1:3 to about 3:1, further preferably from about 1:2 to about 2:1 and very particularly preferably from about 1:1.5 to about 1.5:1.

With a ratio (A)/(B) of 1:2, one part by weight of preparation (A) is mixed with 2 parts by weight of preparation (B).

To produce the mixture, for example, the preparation (A) can be transferred completely from the first container to the second container—which already contains the preparation (B). In this case, the size of the second container is chosen so that it can hold the total quantity of preparations (A) and (B) and allows mixing of preparations (A) and (B), e.g. by shaking or stirring.

Similarly, the mixture can also be produced by completely transferring the preparation (B) from the second container to the first container—which already contains the preparation (A). In this case, the size of the first container should be such that it can hold the total quantity of preparations (A) and (B) and also permits mixing of the two agents (a) and (b), e.g. by shaking or stirring.

If the multi-component packaging unit as contemplated herein comprises the three preparations (A), (B) and (C), the ready-to-use colorant can be prepared by mixing the preparations (A) and (B) and (C), for example in a mixing ratio of about 1:1:1 or about 2:1:1 or about 1:1:2 or about 1:2:1.

Another possibility for producing an appropriate application mixture is the complete transfer of both preparations (A) and (B) from their respective containers into a third container. It may contain the third container, which contains a preparation (C) with at least one coloring compound.

### Process for Dyeing Keratinous Fibers

The user or hairdresser may use the above described preparations (A), (B)—and if applicable (C)—of the multi-component packaging unit as contemplated herein in a process for coloring hair.

A second subject of the present disclosure is a process for dyeing human hair comprising the following steps in the order indicated

(1) Providing a cosmetic preparation (A) as already disclosed in detail when the first subject matter of the present disclosure was described,
(2) Provision of a cosmetic preparation (B) as already disclosed in detail when the first subject-matter of the present disclosure was described,
(3) Mixing of cosmetic preparations (A) and (B),
(4) Application of the application mixture prepared in step (3) to the hair,
(5) Let the application mixture act on the hair and
(6) Rinse off the application mixture.

In other words, a second subject of the present disclosure is a process for dyeing human hair, comprising the following steps in the order indicated

(1) Providing a cosmetic preparation (A) containing at least one organic silicon compound of formula (I)

(R₁O)ₐ(R₂)_{b}Si-(A)_{c}-[NR₃-(A′)]_{d}-[O-(A″)]ₑ-[NR₄-(A′″)]_{f}-Si(R₂′)_{b′}(OR₁′)_{a′} (I),

where
R1, R1′, R1″, R2, R2′ and R2″ independently of one another represent a C₁-C₆ alkyl group,
A, A′, A″, A′″ und A″″ independently of one another represent a linear or branched divalent C₁-C₂₀ alkylene group
R₃ and R₄ independently represent a hydrogen atom, a C₁-C₆ alkyl group, a hydroxy C₁-C₆ alkyl group, a C₂-C₆ alkenyl group, an amino C₁-C₆ alkyl group or a group of formula (II)

(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″,
c is 0 or 1,
d is 0 or 1,
e stands for 0 or 1,
f stands for 0 or 1,
provided that at least one of c, d, e, and f is different from 0,
   (2) Provision of a cosmetic preparation containing water (B),
   (3) Mixing of cosmetic preparations (A) and (B),
   (4) Application of the application mixture prepared in step (3) to the hair,
   (5) Let the application mixture act on the hair and
   (6) Rinse off the application mixture.

The technical application properties of the resulting dyeing can be further improved by selecting the optimum process conditions.

It has been found that the duration of the mixing process can have a significant influence on the color result. If the user mixes the two preparations (A) and (B) for too short a period of time, the viscosity of the application mixture necessary for the application has not yet built up to a sufficient degree, so that the application mixture drips off the keratin fibers in an undesirable way. However, if the user mixes the two preparations (A) and (B) for too long, the polymerization is largely completed before the application mixture meets the hair. In this case the formation of a uniform, dye-containing film on the hair is made more difficult. Against this background, it has proven to be particularly preferred if the user mixes the cosmetic preparations (A) and (B) manually (e.g. by shaking or stirring) for a period of from about 10 seconds to about 10 minutes, preferably from about 20 seconds to about 5 minutes and particularly preferably from about 30 seconds to about 3 minutes.

In the context of a further form of execution, a method is therefore particularly preferred, exemplified by the

(3) manual mixing of cosmetic preparations (A) and (B) for a period of from about 10 seconds to about 10 minutes, preferably from about 20 seconds to about 5 minutes and particularly preferably from about 30 seconds to about 3 minutes.

An analogous influence on the technical application properties of the coloring can be exerted by the period that exists between the preparation of the application mixture and the application of this application mixture to the hair.

If the user applies the application mixture too early after its production, the viscosity is not yet sufficiently high so that the application mixture drips off the keratin fibers in an undesired way. However, if the user waits too long, the polymerization is already largely complete. The “in situ” formation of the dye-containing silicone films, which are the prerequisite for the outstanding fastness properties of this dyeing principle, is no longer possible in this way.

In the context of a further form of execution a procedure is therefore particularly preferred,

exemplified by the
(4) Application of the application mixture prepared in step (3) to the hair within a period of about 30 minutes, preferably about 20 minutes, further preferably about 15 minutes and particularly preferably about 10 minutes after its preparation.

The process as contemplated herein allows the production of dyeing's with particularly good intensity and wash fastness even with short exposure times. Application times from about 10 seconds to about 10 minutes, preferably from about 20 seconds to about 5 minutes and most preferably from about 30 seconds to about 2 minutes on the hair have proven to be particularly beneficial.

In the context of a further form of execution a procedure is therefore particularly preferred,

exemplified by the
(5) Allow the application mixture to act on the hair for a period of from about 10 seconds to about 10 minutes, preferably from about 20 seconds to about 5 minutes and most preferably from about 30 seconds to about 2 minutes.

Particularly advantageous results could be obtained if the application mixture prepared in step (3) of the process as contemplated herein contains the organic silicon compound(s) of formula (I) in an amount of from about 0.5 to about 30.0% by weight, preferably from about 1.0 to about 25.0% by weight and most preferably from about 5.0 to about 20.0% by weight.

In the context of a further form of execution a procedure is therefore particularly preferred, exemplified in that the application mixture prepared in step (3)—based on its total weight—(a) contains one or more organic silicon compounds corresponding to formula (I) in a total quantity of from about 0.5 to about 30.0% by weight, preferably from about 1.0 to about 25.0% by weight and very particularly preferably from about 5.0 to about 20.0% by weight.

In the context of a further form of execution a procedure is particularly preferred, exemplified in that the application mixture prepared in step (3)—based on its total weight—(b) from about 30 to about 95% by weight −5, preferably from about 40 to about 90% by weight and particularly preferably from about 50 to about 90% by weight of water.

In the context of a further form of execution a procedure is therefore particularly preferred,

exemplified in that the application mixture prepared in step (3) contains the organic silicon compound(s) of formula (I) and water in a weight ratio of from about 1:10 to about 1:1, preferably from about 1:7 to about 1:1, further preferably from about 1:6 to about 1:2 and very particularly preferably from about 1:5 to about 1:3.

Concerning the other preferred embodiments of the method as contemplated herein, the same applies mutatis mutandis to the multi-component packaging unit as contemplated herein.

### EXAMPLES

### 1. Formulations

The following formulations were produced.

### Preparation (A)

| | (A1) | (A2) | (A3) |
|---|---|---|---|
| (3-Aminopropyl)triethoxysilan | 20 g | — | — |
| 3-(Triethoxysilyl)-N-[3-(triethoxysilyl)propyl]-1-propanamine | — | 20 g | — |
| N-methyl-3-(triethoxysilyl)-N-[3-(triethoxysilyl)propyl]-1-propanamine | — | — | 20 g |

| | | |
|---|---|---|
| (B1) | (B2) | (B3) |

| | | | | | | |
|---|---|---|---|---|---|---|
| Cocoamidopropyl | 1.5 | g | 1.5 | g | 1.5 | g |
| betaine | | | | | | |

| | | | | |
|---|---|---|---|---|
| Ext. D&C Violet 2 | 1.0 | g | — | — |
| (Acid Violet 43, | | | | |
| CI 60730, | | | | |
| CAS-No. 4430-18-6) | | | | |

| | | | | |
|---|---|---|---|---|
| Basic Red 51 | — | 0.5 | g | — |
| (CAS-No. 77061-58-6) | | | | |

| | | | | |
|---|---|---|---|---|
| Xirona Magic | — | — | 4.5 | g |
| Mauve Pigment | | | | |
| (Merck) | | | | |
| Silica, CI 77891 | | | | |
| (Titanium Dioxide), | | | | |
| Tin Oxide | | | | |

| | | | |
|---|---|---|---|
| Citric acid | ad pH 3.5 | — | — |
| Lactic acid | — | ad pH 3.5 | — |
| Sulfuric acid | — | — | ad pH 3.5 |

| | | | | | | |
|---|---|---|---|---|---|---|
| Water | ad 80 | g | ad 80 | g | ad 80 | g |

### 2. Application

The preparations (A) and (B) were each shaken together for the specified period and then left to stand at room temperature for the specified period.

Each of the application mixtures was then applied with a brush to hair strands (Kerling 6-0) and left to work for the specified period. Afterwards the hair strands were first washed out with water and then washed and dried with a shampoo. Afterwards the strands were visually evaluated.

The strands were then washed several times with a shampoo and again visually evaluated.

| | V1 | E1 | E2 |
|---|---|---|---|
| | 20 g (A1) + | 20 g (A2) + | 20 g (A3) + |
| | 80 g (B1) | 80 g (B1) | 80 g (B1) |
| Mixing | 3 min | 3 min | 3 min |
| Standing time after | 5 min | 5 min | 5 min |
| mixing | | | |
| Application time | 5 min | 5 min | 5 min |
| Result | violet coloration | violet coloration | violet coloration |
| | ++ | +++ | +++ |
| | particularly good | particularly good | particularly good |
| | wash ability | washability | washability |

V = Comparison
E = according to present disclosure
Intensity: + bad ++ medium +++ high

| | V2 | | E3 | E4 |
|---|---|---|---|---|
| | 20 g (A1) + | | 20 g (A2) + | 20 g (A3) + |
| | 80 g (B2) | | 80 g (B2) | 80 g (B2) |
| Mixing | 3 | min | 3 min | 3 min |
| Standing time after | 60 | min | 5 min | 5 min |

| mixing | | | |
|---|---|---|---|
| Application time | — | 5 min | 5 min |
| Result | Application mixture | red coloration | red coloration |
| | extremely high | +++ | +++ |
| | viscosity | particularly good | particularly good |
| | Application to the | washability | washability |
| | hair strand not | | |
| | possible | | |

V = Comparison
E = according to present disclosure

| | V3 | | E5 | E6 |
|---|---|---|---|---|
| | 20 g (A1) + | | 20 g (A2) + | 20 g (A3) + |
| | 80 g (B3) | | 80 g (B3) | 80 g (B3) |
| Mixing | 3 | min | 3 min | 3 min |
| Standing time after | 5 | min | 5 min | 5 min |
| mixing | | | | |
| Application time | 60 | min | 5 min | 5 min |

| | | | |
|---|---|---|---|
| Result | Application mixture | metallic violet | Metallic violet |
| | extremely high | coloring | coloration |
| | viscosity | +++ | +++ |
| | poor removability | particularly good | particularly good |
| | from the strand | washability | washability |

V = Comparison
E = according to present disclosure

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Multi-component packaging unit for dyeing keratinous fibers, which comprises separately from one another
a first container comprising a first cosmetic preparation (A) and
a second container comprising a second cosmetic preparation (B),
where
the first cosmetic preparation (A) comprises at least one organic silicon compound corresponding to formula (I)
(R₁O)ₐ(R₂)_{b}Si-(A)_{c}-[NR₃-(A′)]_{d}-[O-(A″)]ₑ-[NR₄-(A′″)]_{f}-Si(R₂′)_{b′}(OR₁′)_{a′} (I),
where
R1, R1′, R1″, R2, R2′ and R2″ independently of one another represent a C₁-C₆ alkyl group,
A, A′, A″, and A′″ independently of one another represent a linear or branched divalent C₁-C₂₀ alkylene group,
R₃ and R₄ independently represent a hydrogen atom, a C₁-C₆ alkyl group, a hydroxy C₁-C₆ alkyl group, a C₂-C₆ alkenyl group, an amino C₁-C₆ alkyl group or a group of formula (II)
(A″″)-Si(R₂″)_{b}″(OR₁″)ₐ″ (II),
a, stands for an integer from 1 to 3,
b stands for the integer 3-a,
a′ stands for an integer from 1 to 3,
b′ stands for the integer 3-a′,
a″ stands for an integer from 1 to 3,
b″ stands for the integer 3-a″,
c is 0 or 1,
d is 0 or 1,
e stands for 0 or 1,
f stands for 0 or 1,
A″″ represents a linear or branched divalent C₁-C₂₀ alkylene group,
provided that at least one of c, d, e, and f is different from 0, and
the second cosmetic preparation (B) comprises water.

2. Multi-component packaging unit according to claim 1, wherein the cosmetic preparation (A) comprises at least one organic silicon compound of formula (I)
where
R1 and R1′ independently represent a methyl group or an ethyl group,
a and a′ both stand for the number 3 and
b and b′ both stand for the number 0.

3. Multi-component packaging unit according to claim 1, wherein the cosmetic preparation (A) comprises at least one organic silicon compound corresponding to formula (I)
where
c and d both stand for the number 1,
e and f both stand for the number 0,
A and A′ independently of one another represent a linear, divalent C₁-C₆ alkylene group
and
R3 represents a hydrogen atom, a methyl group, a 2-hydroxyethyl group, a 2-alkenyl group, a 2-aminoethyl group or a group of formula (II).

4. Multi-component packaging unit according to claim 1, wherein the cosmetic preparation (A) comprises at least one organic silicon compound of the formula (I) which is selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

5. Multi-component packaging unit according to claim 1, wherein the cosmetic preparation (A) comprises—based on the total weight of the preparation (A)—one or more organic silicon compounds of the formula (I) in a total amount of from about 20 to about 100% by weight.

6. Multi-component packaging unit according to claim 1, wherein the first cosmetic preparation (A) is substantially anhydrous.

7. Multi-component packaging unit according to claim 1, wherein the cosmetic preparation (A) comprises one or more fat constituents chosen from the group of C₈-C₃₀ fatty alcohols, C₈-C₃₀ fatty acid triglycerides, C₈-C₃₀ fatty acid monoglycerides, C₈-C₃₀ fatty acid diglycerides, C₈-C₃₀ fatty acid esters and/or hydrocarbons.

8. Multi-component packaging unit according to claim 1, wherein the first cosmetic preparation (A) comprises at least one colorant compound chosen from the group of direct dyes and/or pigments.

9. Multi-component packaging unit according to claim 1, wherein the second cosmetic preparation (B) comprises—based on the total weight of the preparation (B)—from about 50 to about 100% by weight of water.

10. Multi-component packaging unit according to claim 1, wherein the second cosmetic preparation (B) comprises at least one acid chosen from the group of lactic acid, citric acid, tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid.

11. Multi-component packaging unit according to claim 1, wherein the second cosmetic preparation (B) has a pH value in the range from about 0.5 to about 6.

12. Multi-component packaging unit according to claim 1, wherein the second cosmetic preparation (B) comprises at least one surfactant chosen from the group of anionic surfactants, nonionic surfactants, zwitterionic surfactants, amphoteric surfactants, and/or cationic surfactants.

13. Multi-component packaging unit according to claim 1, wherein the second cosmetic preparation (B) comprises at least one colorant compound chosen from the group of direct dyes and/or pigments.

14. A method for dyeing human hair comprising the following steps in the order indicated
(1) providing a cosmetic preparation (A) as described in claim 1,
(2) providing a cosmetic preparation (B) as described in claim 1,
(3) mixing cosmetic preparations (A) and (B),
(4) applying the application mixture prepared in step (3) to the hair,
(5) allowing the application mixture act on the hair and
(6) rinsing off the application mixture.

15. Process according to claim 14, wherein
(3) cosmetic preparations (A) and (B) are manually mixed for a period of from about 10 seconds to about 10 minutes.

16. Process according to claim 14, wherein
(4) the application mixture prepared in step (3) is applied to the hair within a period of 30 minutes.

17. Process according to claim 14 wherein
(5) the application mixture is allowed to act on the hair for a period of from about 10 seconds to about 10 minutes.

18. Process according to claim 14, wherein the application mixture prepared in step (3)—based on its total weight—
(a) comprises one or more organic silicon compounds corresponding to formula (I) in a total quantity of from about 0.5 to about 30.0% by weight.

19. Process according to claim 14, wherein the application mixture prepared in step (3)—based on its total weight—
(b) comprises from about 30 to about 95% of water.

20. Process according to claim 14, wherein the application mixture prepared in step (3) comprises the organic silicon compound(s) of the formula (I) and water in a weight ratio of from about 1:10 to about 1:1.
